(19) United States
(12) Patent Application Publication
(10) Pub. No.: US 2010/0332250 A1
(43) Pub. Date: Dec. 30, 2010

(54) PRE-EXAMINATION MEDICAL DATA ACQUISITION SYSTEM

(75) Inventors: Mark R. Simpson, White Plains, NY (US); Lalitha Agnihotri, Hartsdale, NY (US); William Tao Shi, Briarcliff Manor, NY (US); James David Schaffer, Wappingers Falls, NY (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., EINDHOVEN (NL)

(21) Appl. No.: 12/747,645

(22) PCT Filed: Dec. 10, 2008
(86) PCT No.: PCT/IB2008/055201

§ 371 Date: Sep. 2, 2010

Related U.S. Application Data
(60) Provisional application No. 61/015,991, filed on Dec. 21, 2007.

(51) Int. Cl. G06Q 50/00; G06Q 30/00; G06Q 90/00; G06F 17/00; G06F 15/16; G06F 17/30; A61B 5/00

(52) U.S. Cl. 705/2; 705/500; 709/203; 707/769; 600/300; 707/E17.014

(57) ABSTRACT

A pre-examination patient information gathering system comprises an electronic user interface (30, 130) including a display (32) and at least one user input device (34, 36), and an electronic processor (50) configured to present an initial set of questions (54) to a patient via the electronic user interface, receive responses to the initial set of questions from the patient via the electronic user interface, construct or select follow-up questions (68) based on the received responses, present the constructed or selected follow up questions to the patient via the electronic user interface, and receive responses to the constructed or selected follow up questions from the patient via the electronic user interface. A physiological sensor (70, 72, 74, 76, 78, 80) may be configured to autonomously sense a patient physiological parameter as the patient interacts with the electronic user interface.

The following relates to the medical arts, medical diagnostic arts, medical administration arts, and the like.

A typical medical office run by a physician or a group of physicians operates on an appointment basis, in which a patient schedules an appointment, arrives at the medical office a few minutes before the appointed time, waits in a waiting room until the appointed time, and is seen by the physician at the appointed time. Ideally, each patient is examined by the physician for an allotted time, and the physician moves efficiently on to the next scheduled patient.

In practice, however, it is known that medical offices sometimes run behind schedule. This can occur when an emergency situation arises due to a critical condition of a patient identified during examination or arriving in the form of an unscheduled “walk-in” patient. However, it is also known that medical offices sometimes run behind schedule even when there is no emergency, because uneventful patient examinations nonetheless sometimes overrun their allotted times. One cause for such overruns is inefficient transfer of patient information to the examining physician.

It is also known that in such a typical medical office, patients arriving to see a physician wait in a waiting room until the physician is available to see them. These waits are not problematic when the medical office is running on time, but can become annoyingly long when the medical office is running behind schedule. Besides inconveniencing patients, a medical office running behind schedule makes for inefficient use of the valuable time of highly skilled physicians.

The medical field has recognized that there is an opportunity presented here to enhance efficiency—the patient can convey relevant information while waiting in the waiting room, so that when the patient is seen by the physician the examination is expedited. A synergy should result—the patient conveys information while waiting in the waiting room, which expedites the examination, which in turn reduces subsequent patients' wait in the waiting room.

Thus, it is known to have a receptionist give each incoming patient a medical information form and a pen, and for the patient to fill out the form with requested medical information. The patient fills out the form and returns it to the receptionist.

Unfortunately, it is known that patient waits remain annoyingly long. Having patients fill out medical information forms does not seem to substantially enhance office efficiency. Although the information is collected, it is not acted upon until the patient examination. Consider an instance in which the patient writes down that he or she is experiencing chest pain. The physician would likely want to follow up with additional information requests: What kind of chest pain? Where is it located? Is it an intermittent sharp stabbing pain, or a dull continuous pain? Is it accompanied by breathing difficulty? But, the physician does not have the opportunity to ask these follow-up questions until the patient examination, and so little or no efficiency gain is realized.

The medical profession has realized this to some extent, though the use of paper forms as a way of gathering pre-examination patient information remains popular. Medical offices have attempted to overcome the information review bottleneck by the use of physician assistants. These are medical personnel who are not physicians, but who have some medical training They may be registered nurses (RN's), physician aides, or so forth. The concept here is that the patient does not wait for the entire time in the waiting room, but rather when the time for examination draws near the patient is escorted into an examination room, typically the room the physician will use for the patient examination. There, the physician assistant acquires patient information. In addition to asking questions, the physician assistant is usually trained to acquire certain physiological parameters, usually vital signs such as pulse, blood pressure, weight, or so forth.

Unfortunately, it is known that patient waits remain annoyingly long. Having patients undergo “pre-examinations” conducted by a physician assistant does not seem to substantially enhance office efficiency. The physician assistant is usually not trained, or at least not authorized, to make substantive decisions based on the results of the pre-examination. For example, if the patient asks the physician assistant a seemingly simple question such as whether a given vital sign reading is good, the physician assistant will commonly defer to the physician to make this determination. Also, these pre-examinations are usually conducted in the same room as the eventual patient examination conducted by the physician. This means that the medical office must have multiple examination rooms, each equipped adequately to perform a complete patient examination. Still further, the physician assistant's time is also valuable, albeit not as valuable as the physician's time.

In summary, there is widespread recognition in the medical profession that long patient waits in the waiting room are bad. In addition to the aforementioned patient inconvenience, the waiting room represents an unfortunate admixture of diverse airborne and surface mediated contagions, and the likelihood that patients will contract contagions from one another increases with longer patient waits. There is further recognition that medical questionnaire forms and pre-examinations conducted by physician assistants, although seemingly promising, do not in practice substantially reduce long patient waits.

The following provides improvements, which overcome the above-referenced problems and others.

In some embodiments disclosed herein as illustrative examples, a pre-examination patient information gathering system is disclosed, comprising: an electronic user interface including a display and at least one user input device; and an electronic processor configured to present an initial set of questions to a patient via the electronic user interface, receive responses to the initial set of questions from the patient via the electronic user interface, construct or select follow-up questions based on the received responses, present the constructed or selected follow-up questions to the patient via the electronic user interface, and receive responses to the constructed or selected follow up questions from the patient via the electronic user interface.

In some embodiments disclosed herein as illustrative examples, a pre-examination patient information gathering method is disclosed, comprising: presenting an initial set of questions to a patient via an electronic user interface including a display and at least one user input device; receiving responses to the initial set of questions from the patient via the electronic user interface; electronically constructing or selecting follow-up questions based on the received responses; presenting the follow-up questions to the patient via the electronic user interface; and receiving responses to the follow-up questions from the patient via the electronic user interface.

In some embodiments disclosed herein as illustrative examples, a pre-examination patient information gathering method is disclosed, comprising: arranging an electronic physiological sensor in a medical office such that the electronic physiological sensor can sense a patient physiological parameter; and autonomously operating the electronic physiological sensor to unobtrusively sense the patient physiological parameter.

In some embodiments disclosed herein as illustrative examples, a pre-examination patient information gathering system is disclosed, comprising: a user interface for presenting the patient with questions and for receiving responses to the questions from the patient; and an electronic physiological sensor arranged to unobtrusively sense a patient physiological parameter while the patient is interacting with the user interface.

One advantage resides in more efficient medical office operation.

Another advantage resides in reduced patient waiting times at medical offices.

Another advantage resides in more efficient use of valuable physician time.

Another advantage resides in the possibility that more thorough information may be collected than might be collected by less experienced medical practitioners. In some cases an automatic sensor may pick up parameters that are difficult or impossible for a human medical practitioner to detect or measure accurately.

Still further advantages of the present invention will be appreciated to those of ordinary skill in the art upon reading and understand the following detailed description.

The drawings are only for purposes of illustrating the preferred embodiments, and are not to be construed as limiting the invention.

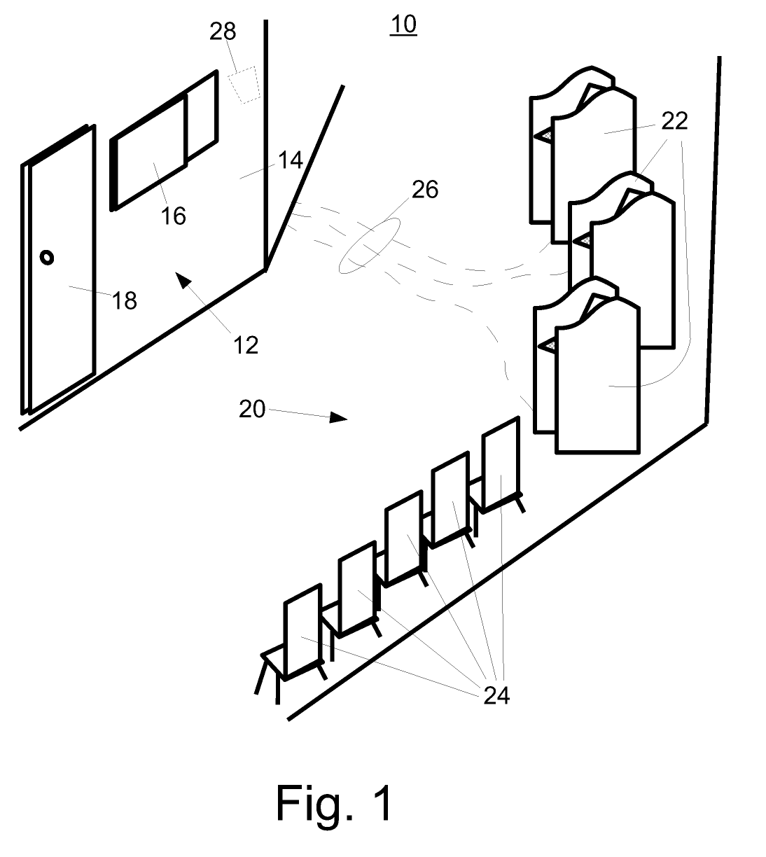

FIG. 1 diagrammatically shows a medical office including kiosks for enabling a patient to provide pre-examination patient information.

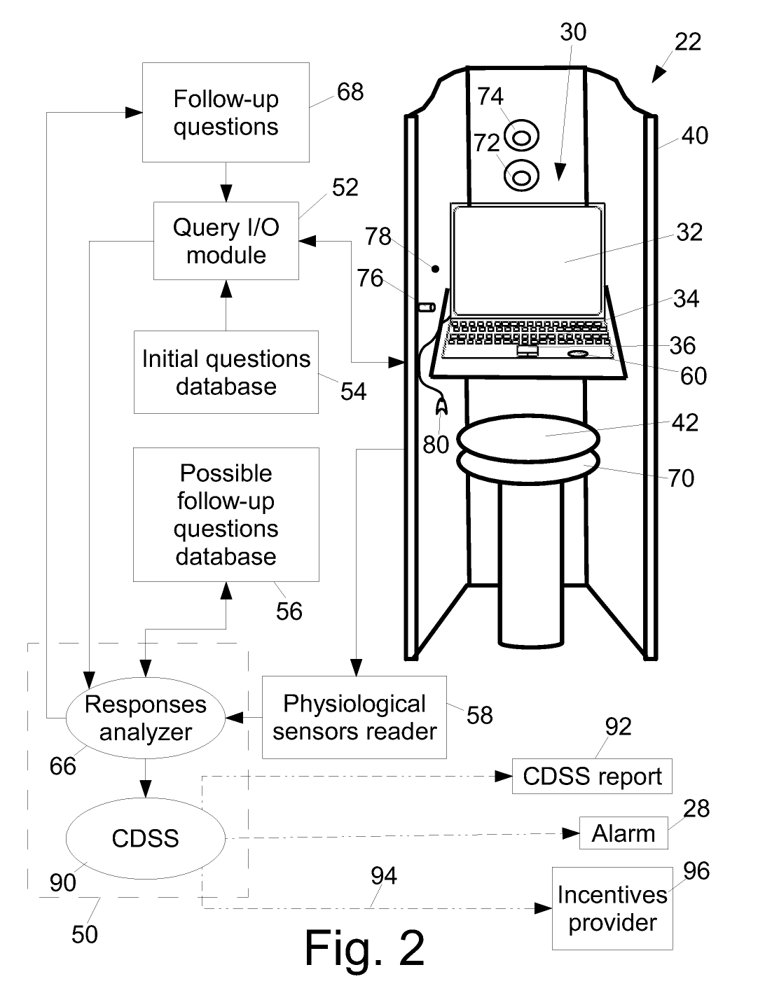

FIG. 2 diagrammatically shows a pre-examination patient information gathering system employing one of the kiosks of FIG. 1.

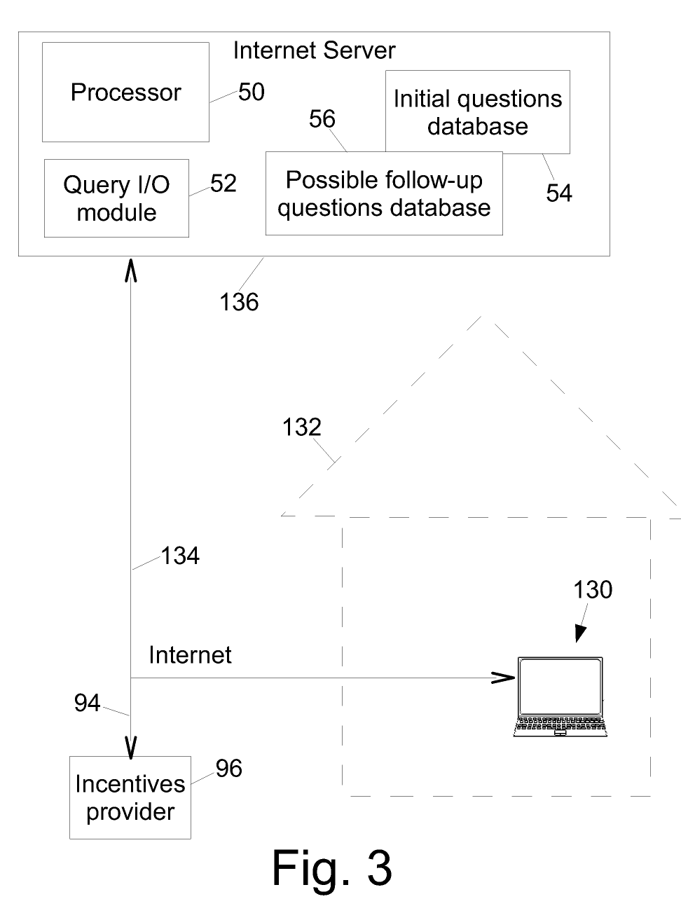

FIG. 3 diagrammatically shows a pre-examination patient information gathering system employing a home computer as the user interface.

With reference to FIG. 1, a medical office 10 includes a patient examination area 12, which in the illustrated embodiment includes a set of one or more discreetly private examinations rooms (not shown) disposed behind a wall or barrier 14 having a receptionist's window 16 and an access door 18. The medical office 10 further includes a waiting area 20 outside of the examination area 12. As used herein, the term “medical office” is intended to encompass any facility at which physicians, doctors, or other medical personnel conduct a patient examination for the purpose of medical diagnosis, clinical evaluation, clinical monitoring, fitness evaluation, or other medically-related purpose. In operation, a patient arrives and reports in with the receptionist at the receptionist's window 16. As used herein, the term “patient” is intended to encompass a person using the pre-examination patient information gathering systems and methods disclosed herein to provide medical information preparatory to a contemplated patient examination. As used herein, the patient may in some instances be suffering from a specific malady or symptom which is to be the subject of the patient examination, or the patient may be in medically good condition and may for example only be coming in for a patient examination comprising a routine general physical examination. In any event, the patient is expected to arrive at least a few minutes before a scheduled patient examination appointment, and is accordingly directed by the receptionist to wait in the waiting area 20.

In order to expedite operation of the medical office, the patient is requested to answer selected pre-examination questions at an available electronic kiosk 22 while waiting in the waiting area 20. In the illustrative embodiment of FIG. 1, three kiosks 22 are provided in the waiting area 20 so as to accommodate up to three patients at the same time; however, the number of provided kiosks can be one, two, three, four, five, or more. The patient interacts with the electronic kiosk 22 to provide responses to the questions and, in some embodiments, to facilitate gathering of other patient information. Once the pre-examination patient information gathering is completed, the patient may sit down at illustrative provided chairs 24, or may elect to stand or otherwise occupy time. When the physician, doctor, or other appropriate medical person is available, the receptionist calls the patient and the patient enters the patient examination area 12 via the door 18, and the physician conducts the patient examination in an examination room or other appropriate portion of the patient examination area 12. In conducting the examination, the physician suitably refers to information gathered from the patient while waiting in the waiting area 20 using one of the electronic kiosks 22.

In a typical medical office, the physician sees patients on a serially scheduled appointment basis, in which the patient examination appointments are scheduled in succession so as to occupy most of the physician's time. In this mode of operation, the physician typically does not consider the information gathered from the patient via the kiosk 22 until the patient is admitted into the patient examination area 12 for the patient examination, or perhaps shortly before such admittance. In some embodiments, however, operative connections 26 are provided linking the kiosks 22 with the patient examination area 12. The illustrated operative connections 26 are wired connections such as wired local area network (LAN) cabling or a dedicated wired connection passing through a floor or other infrastructure of the medical office 10; however, wireless operative connections are also contemplated, such as a wireless local area network (WLAN) connection or a dedicated wireless transmitter/receiver combination. The electronic kiosks 22 in these embodiments have autonomy sufficient to recognize certain conditions that may represent an emergency medical condition, a highly contagious condition, or some other recognizable condition suggesting that the patient should be seen immediately or moved up in the schedule. When the kiosk 22 detects such a condition, a suitable signal, data set, or other information is conveyed via the operative connections 26 to the patient examination area 12 where a suitable alarm 28 is activated. The alarm 28 may be a light, sign, or display that is visible from within the patient examination area 12 but is not visible from the waiting area 20, so that the receptionist or other personnel in the patient examination area 12 are notified of the condition. Alternatively or additionally, the alarm 28 can include an audible alarm, a pop-up window on a computer (not shown) operated by the receptionist, or other suitable alarm. The physician is notified of the alarm condition and can take suitable action, such as calling the patient in for an immediate patient examination, or isolating the patient if the alarm condition indicates the patient is highly contagious, or so forth.

With reference to FIG. 2, operation of the pre-examination patient information gathering system is described with respect to an illustrative one of the electronic kiosks 22. The illustrated kiosk 22 includes a user interface 30, which may for example be a computer or terminal having a display 32 and at least one user input device such as a keyboard 34, touch-pad 36, or so forth. A privacy enclosure or curtain 40 is provided to ensure that the patient's responses to presented questions remain confidential or private. In the illustrated embodiment, the patient sits at a stool 42. Alternatively, another piece of furniture could be provided, or the patient could stand at the kiosk 22. The shown kiosk 22 is an illustrative example, and diverse variations are contemplated. For example, the kiosk can be a cubicle or small room with a desk supporting a computer or computer terminal, or a laptop computer, tablet computer, or the like used by the patient sitting at one of the chairs 24, or so forth. The term “kiosk” as used herein is intended to encompass any apparatus including an electronic user interface located at the medical office (10) and configured for use by a patient to provide pre-examination patient information.

An electronic processor 50 controls the pre-examination patient information process. The processor is diagrammatically indicated in FIG. 2, and may be variously embodied as suitable software running on the illustrated computer-embodied user interface 30, a computer or server located in the patient examination area 12 (not shown) and linked with the kiosk 22 via the operative connection 26, a web-based server, or so forth. A query input/output (I/O) module 52 interfaces between the electronic processor 50 and the user interface 30 to format questions for presentation to the patient and to convey received responses back to the processor 50. The query I/O module 52 can also be variously embodied, for example as software running on the illustrated computer-embodied user interface 30, software running on or with the processor 50, or so forth. In some embodiments the user interface 30, processor 50, and query I/O module 52 may be integrated as a single unit, for example embodied as a singular computer running suitable software.

Other components optionally included in the pre-examination patient information gathering system include a database of initial questions 54, a database of follow-up questions 56 (which may or may not be integrated with the initial questions database 54), and a physiological sensors reader 58. Again, each of the components 54, 56, 58 may be variously embodied. For example, the databases 54, 56 may be a storage medium component (e.g., a hard disk, optical disk, floppy disk, solid state memory, remote server, or so forth) of the user interface 30 or electronic processor 50, or may be a stand-alone or other separate storage unit, that stores the initial questions and follow-up questions for presentation to the patient via the user interface 30. The optional physiological sensors reader 58 may be a stand-alone hardware unit, a suitable data acquisition card or communications interface integrated with the user interface 30 or with processor 50, or so forth. Still further, a fingerprint reader 60 or other biometric identification device may be provided to receive or confirm identification of the patient.

In operation of the illustrated pre-examination patient information gathering system, the query I/O module 52 initially operates in conjunction with the user interface 30 to present the initial questions stored in the database of initial questions 54 to the patient. The initial questions are those intended for presentation to all patients regardless of their medical condition or other factors. Some example initial questions may include requests for patient name, address, medical insurance carrier, medical insurance policy number, any drug allergy information, medical history questions, or so forth. Optionally, the questions may be presented as hyperlinks such that if the user clicks on a question using a mouse pointer or the like then a pop-up window provides further explanation or information regarding the question. The query I/O module 52 optionally validates responses. For example, the question “What is your height?” may be validated based on a range, with a height of less than three feet or more than eight feet producing a validation error. The fingerprint reader 60 or other biometric identification device may optionally be used to validate a patient identification response (e.g., an input patient name). Additionally or alternatively, the query I/O interface 52 may configure presentation of some questions to limit responses to an acceptable format. For example, a question answerable by “yes” or “no” may be presented with checkboxes clickable to select the “yes” or “no” answer, so that the user cannot enter anything other than “yes” or “no”. Similarly, a question regarding drug allergies may present a list of common drug allergies with selection checkboxes, optionally with an additional checkbox for “Other” that causes generation of an input line for inputting any unlisted drug allergy the patient may have. Optionally, the query I/O module 52 may access an existing patient database (not shown) and populate the presented questions with default answers corresponding to information already stored in the database. For example, the patient's address and medical insurance information currently on file may be provided as default responses.

The responses received from the patient are input to a responses analyzer 66 of the processor 50 for analysis, optionally after validation by the query I/O module 52. The analysis determines whether one or more follow-up questions should be asked. For example, if the patient responds with “yes” to a question about numbness in the limbs (indicating that the patient indeed is experiencing such numbness) and the patient also responds affirmatively in response to a question as to whether the patient is allergic to a substance “X”, then the responses analyzer 66 recognizes that numbness is an indication of an allergic reaction to substance “X” and further recognizes that the patient is allergic to substance “X” and accordingly constructs or retrieves from the possible follow-up questions database 56 a set of such follow-up questions relating to possible pathways for exposure to substance “X”. For example, the follow-up questions may related to whether the patient has consumed certain foods known to contain substance “X”.

One suitable approach for enabling selection of follow-up questions based on the received responses is as follows. In the database 56, each possible follow-up question is tagged with responses or combinations of responses that should cause selection of the possible follow-up question for presentation as a follow-up question. For example, the initial questions may include:

Q11: Are you currently experiencing chest pains?

Q12: Are you dizzy?

Q13: Do you have numbness in your arms or legs?

The possible follow-up questions database 56 may include the follow-up question:

Q122: Are you experiencing heart palpitations? [Q11=Yes, Q12=Yes & Q13=Yes]

where the tag [Q11=Yes, Q12=Yes & Q13=Yes] indicates that the possible follow-up Question Q122 from the database 56 should be selected for presentation as a follow-up question if the answer to Question Q11 is “Yes” or (the comma indicating disjunction in the illustrative tag notation) if the answer to Questions Q2 and Q3 are both “Yes”. Otherwise, the question Q122 is not selected for presentation as a follow-up question. The tags identifying responses or response combinations that should prompt presentation of a given possible follow-up question are suitably selected by physicians or other medical professionals (possibly in conjunction with assistance of a computer programmer or other technical personnel) so that the follow-up questions presented based on the tags mimic the follow-up questions that would likely be asked by a physician.

With continuing reference to FIG. 2, the responses analyzer 66 analyzes the received responses as described, and constructs or selects follow-up questions 68 that are presented to the user via the user interface 30. Optionally, this processing may be repeated for one, two, three, or more iterations. For example, based on responses received for certain follow-up questions, a search of tags of the follow-up questions database 56 may cause selection of further follow-up questions for presentation to the patient. It should also be appreciated that the tag-based combinational selection process is one suitable embodiment, but that other approaches for constructing or selecting follow-up questions can also be used. For example, the possible follow-up questions may be organized in the database 56 according to question classification, and a certain class of possible follow-up questions may be selected by a particular received response. For example, a class of questions relating to cardiac conditions may be selected upon receipt of a response indicating that the patient has a family history of heart disease.

The disclosed approach of selectively presenting follow-up questions based on received responses advantageously reduces the total number of questions presented to the patient by avoiding presentation of questions that are not relevant to the patient. This approach expedites the pre-examination patient information gathering process when compared with paper forms that do not provide such selective questioning, and also advantageously avoids confusing patients by presenting irrelevant questions.

Optionally, the pre-examination patient information gathering system also includes one or more physiological sensors configured to sense patient physiological parameters. The physiological sensors are suitably arranged in or with the kiosk 22 to sense the patient physiological parameter as the patient interacts with the at least one user input device 34, 36. In the illustrative example, physiological sensors include a patient scale 70 integrated into the stool 42 such that the patient weight is unobtrusively and autonomously measured when the patent sits down at the kiosk 22. If the patient stands at the kiosk, then a patient weight scale can similarly be integrated into the floor where the patient stands. As another example, an infrared camera 72 can be arranged to image the patient sitting at the kiosk 22, and the physiological sensors reader 58 configured to analyze the infrared image to detect abnormal body temperature regions or patterns that may be indicative of blood circulation difficulties or other medical conditions. Similarly, a visible light camera 74 can be arranged to image the patient sitting at the kiosk 22, and the physiological sensors reader 58 configured to analyze the visible light image to detect abnormal body motions that may be indicative of Parkinson's disease or medical conditions. A chemical sensor 76 can be arranged to detect an airborne chemical emanating from the patient (e.g. breathalyzer) sitting at the kiosk 22, such as a chemical used in chemotherapy or radiation therapy, alcohol indicating possible drunkenness, or so forth. Similarly, chemical sensors integrated into the keyboard 34 or touch-pad 36 may be configured to detect a chemical transmitted by contact.

It will be appreciated that the physiological sensors 70, 72, 74, 76 are autonomous, unobtrusive sensors that sense the desired physiological condition unobtrusively and autonomously while the patient is interacting with the user interface 30. By “autonomous” it is meant that the sensors operate without action taken by personnel of the medical office 10, except perhaps for initializing actions such as turning on the power for the sensors reader 58. By “unobtrusive” it is meant that the sensor detects the condition without requiring affirmative action by the patient directed toward the sensing. For example, the patient sits at the kiosk 22 and inputs responses to presented questions. These are actions on the part of the patient, and they are actions that may facilitate the sensing by placing the patient in proximity to the sensor, but these actions are not directed toward the sensing but rather are directed toward inputting responses to presented questions.

In addition, some physiological sensors may operate autonomously but not unobtrusively. For example, a microphone 78 can be arranged to detect language difficulties such as slurred speech that may be indicative of drunkenness. The user is prompted by a message on the display 32 to say a selected verbiage, such as to orally count from one to ten. Thus, the sensing is not unobtrusive because it entails affirmative action by the patient (oral counting) directed toward the sensing. On the other hand, if the user interface is configured to receive responses to presented questions orally, and the microphone 78 is used to monitor these responses, then the microphone 78 qualifies as an unobtrusive sensor, because in this case the verbiage is not directed toward the sensing but rather toward providing responses for the presented questions.

As another example of a physiological sensor that is not unobtrusive, a fingertip SpO₂ sensor 80 may be arranged for use by the patient. Again, user action direct to the sensing is involved, in that the user inserts the fingertip SpO₂ sensor 80 over the fingertip in order for pulse rate and blood oxygenation sensing to occur.

The pre-examination patient information gathering system collects patient information in the form of received responses to presented questions, and optionally also in the form of physiological parameters sensed autonomously and optionally unobtrusively. In some embodiments, this information is collected, stored (for example in the electronic patient records database of the medical office 10), and presented to the physician at or before the patient examination, but the collected information is not further processed.

With continuing reference to FIG. 2, in some embodiments the collected information is further processed. In the illustrative example, the electronic processor 50 is further configured to define a clinical decision support system (CDSS) 90 configured to generate clinical support content based on the received responses and optionally on sensed physiological parameters. The CDSS 90 may be configured, for example, as an inference engine that infers the possible existence of a medical condition based on a received response or sensed physiological parameter, or based on a combination of received responses, sensed physiological parameters, or both. For example, in the aforementioned example of a patient with an allergy to substance “X”, the CDSS 90 may infer that the patient is suffering from an allergic reaction to exposure to substance “X” based on (i) the patient affirmatively indicating an allergy to substance “X” and (ii) the patient affirmatively indicating experiencing numbness (which in this example is an indication of an allergic reaction to substance “X”) and (iii) indicating in response to a follow-up question that the patient has consumed food known to contain substance “X”. It will be appreciated that although the responses analyzer 66 and CDSS 90 are shown in FIG. 2 as separate components, in practice these components may be integrated. For example, if the inference engine of the CDSS 90 recognizes that it needs a certain datum in order to accept or reject an inference, it suitably causes the responses analyzer 66 to select or construct a follow-up question for presentation configured to solicit a response from the patient providing that datum.

Based on the inferences drawn by the optional CDSS 90, a CDSS report 92 is optionally generated which provides the physician with a summary of the drawn inference or inferences in a readable English format, as a tabulation, or in another format or combination of formats comprehensible by the physician. Optionally, if the drawn inference suggests that some urgent action might be appropriate, the CDSS 90 can activate the alarm 28 located in the patient examination area 12.

The disclosed pre-examination patient information gathering systems and methods have substantial advantages over existing approaches using paper questionnaire forms, pre-examination by a nurse or other physician assistant, or so forth. The kiosks 22 are located outside of the patient examination area 12 and in the patient waiting area 20, and therefore do not occupy valuable and well-equipped patient examination rooms. No nurse or other physician assistant is utilized, which reduces cost and allows these valuable medical professionals to perform other tasks. The patient is not annoyed by being asked duplicative questions on a series of forms, or by being asked irrelevant questions. The use of follow-up questions constructed or selected based on received responses ensures that the gathered pre-examination patient information is probative of the patient. This also ensures that the physician is presented with relevant information, whereas a physician reviewing a paper form completed by the patient receives a substantial number of irrelevant questions and responses from which must be identified those (typically relatively few) responses that are most relevant for evaluating the patient undergoing examination. Still further, since the responses are received in electronic form from the patient via the user interface 30, they are readily compiled and stored in the electronic patient record. In addition, since the opportunity is readily available to include into this setup, automatic acquisition of parameters that may be difficult (requiring expert diagnosticians) or impossible (not directly observable by humans), then more inclusive and insightful data may be collected.

In spite of these substantial advantages, a difficulty recognized herein is that some patients may be reluctant to use the kiosks 22 to provide pre-examination patient information. One way to reduce this reluctance is to make the process more efficient, for example by populating the presented questions with default answers drawn from electronic patient medical records, and by the responses analyzer 66 selecting follow-up questions based on previously received responses so that the patient is not called upon to respond to numerous irrelevant questions, and by having physiological sensors 70, 72, 74, 76 arranged to unobtrusively sense selected physiological parameters of the patient without entailing patient action directed toward the sensing. Nonetheless, it is recognized herein that some patients may be reluctant to use the kiosks 22 even in spite of these efficiency advantages.

Accordingly, in some embodiments a link 94 of the kiosk 22 with an incentives provider 96 is configured to cause the incentives provider 96 to provide an incentive responsive to a patient interaction with the pre-examination patient information gathering system. For example, the link 94 can be via the Internet with a medical insurance company, and configured to cause the insurance company to provide a pecuniary discount responsive to the patient completing an information gathering session using the pre-examination patient information gathering system. Additionally or alternatively, the link 94 can be with a marking engine, for example a printer (not shown) located in the medical office 10, and configured to cause the marking engine to generate a printed coupon redeemable for a pecuniary benefit upon completion of an information gathering session. The coupon can be redeemable for non-medically related subject matter, such as groceries or gasoline, or can be for medically related subject matter such as medical equipment (e.g., glucose monitoring equipment for diabetic patients) or prescription drug refills. As another example, the link can be with the receptionist in the patient examination area 12 (for example, via the operative connection 26), and the receptionist or other office manager operates as the incentives provider 96 by providing the incentive of a reduced waiting time for the patient if the patient utilizes the pre-examination patient information gathering system to provide patient information.

In the embodiments described with reference to FIGS. 1 and 2, the user interface 30 of the pre-examination patient information gathering system is located at the medical office 10, and is used by the patient upon arrival at the medical office 10 for a scheduled patient examination. However, in other embodiments the user interface may be otherwise located.

With reference to FIG. 3, in another embodiment a user interface 130 is embodied as a home computer disposed in the patient's home 132 and connected via the Internet 134 with the processor 50, query I/O module 52, and the databases 54, 56 which are in this embodiment disposed on an Internet server 136 accessible via the Internet 134. The Internet server 136 may be disposed at the medical office 10 of FIG. 1, or may be accessed by the medical office 10 via the Internet 134. Similarly, the optional link 94 with the optional incentives provider 96 is suitably via the Internet 134.

In the embodiment of FIG. 3, the patient accesses the pre-examination patient information gathering system using the home computer 130 as the user interface. In this embodiment, it is to be appreciated that the term “patient” denotes a person planning or contemplating a visit to the medical office 10 for a patient examination. In some embodiments comporting with the arrangement of FIG. 3, it is contemplated that the CDSS 90 may draw an inference that the patient does not need to come into the medical office 10 for a physical examination; such a person is still deemed a “patient” as used herein since the patient is using the pre-examination patient information gathering system to provide medical information preparatory to a contemplated patient examination. The decision reached by the CDSS 90 as to whether the patient should schedule a patient examination at the medical office 10 is suitably communicated to the patient via the home computer 130.

In some embodiments, the CDSS 90 provides an evaluation that is considered by the medical office 10 as a factor in determining how soon the patient should be scheduled for a patient examination. For example, if the CDSS 90 indicates an urgent condition (for example, of the type that would set of the alarm 28 in the embodiments of FIGS. 1 and 2) then the medical office 10 is biased toward scheduling the patient for an immediate appointment or an appointment as soon as practicable. (In some embodiments in which the inference is of a critically urgent condition, it is contemplated for the CDSS 90 to activate the alarm 28 in the form of a message to an emergency medical service calling for an ambulance to be sent immediately to the patient's home 132). On the other hand, if the CDSS 90 make an inference that the condition is not critical (e.g., a skin rash with no other symptoms) then the medical office 10 is suitably biased toward scheduling the patient for an appointment in a non-critical fashion, for example at the next conveniently available appointment slot.

The preferred embodiments have been described. Modifications and alterations may occur to others upon reading and understanding the preceding detailed description. It is intended that the invention be construed as including all such modifications and alterations insofar as they come within the scope of the appended claims or the equivalents thereof.

Having thus described the preferred embodiments, the invention is now claimed to be:

1. A pre-examination patient information gathering system comprising:
an electronic user interface (30, 130) including a display (32) and at least one user input device (34, 36); and
an electronic processor (50) configured to present an initial set of questions (54) to a patient via the electronic user interface, receive responses to the initial set of questions from the patient via the electronic user interface, construct or select follow-up questions (68) based on the received responses, present the constructed or selected follow-up questions to the patient via the electronic user interface, and receive responses to the constructed or selected follow-up questions from the patient via the electronic user interface.

2. The pre-examination patient information gathering system as set forth in claim 1, further comprising:
a link (94) with an incentives provider (96) configured to cause the incentives provider to provide an incentive responsive to a selected patient interaction with the pre-examination patient information gathering system.

3. The pre-examination patient information gathering system as set forth in claim 2, wherein the link (94) with the incentives provider (96) includes at least one of:
a link with a company configured to cause the company to provide a pecuniary discount responsive to the patient completing an information gathering session using the pre-examination patient information gathering system;
a link with a marking engine configured to cause the marking engine to generate a printed coupon redeemable for a pecuniary benefit responsive to the patient completing an information gathering session using the pre-examination patient information gathering system; and
expedite scheduling to visit a medical provider.

4. The pre-examination patient information gathering system as set forth in claim 1, further comprising:
a kiosk (22) including the electronic user interface (30) disposed in a medical office (10).

5. The pre-examination patient information gathering system as set forth in claim 4, wherein the kiosk (22) includes a privacy enclosure or curtain (40).

6. The pre-examination patient information gathering system as set forth in claim 4, further comprising:
an alarm (28) configured to alert the medical office (10) conditional upon a received response or a combination of received responses satisfying an alarm condition.

7. The pre-examination patient information gathering system as set forth in claim 4, wherein the electronic user interface (30) further comprises:
a physiological sensor (70, 72, 74, 76, 78, 80) configured to autonomously sense a patient physiological parameter, the physiological sensor being arranged in or with the kiosk (22) to sense the patient physiological parameter as the patient interacts with the electronic user interface.

8. The pre-examination patient information gathering system as set forth in claim 1, further comprising:
an Internet server (136) configured to operatively connect the electronic user interface (130) and the electronic processor (50) via the Internet (134).

9. The pre-examination patient information gathering system as set forth in claim 8, wherein the electronic processor (50) is further configured to generate a decision as to whether or not the patient should schedule a visit to a medical office (10) or how promptly the visit should be scheduled based on the received responses and to communicate the decision to the patient via the electronic user interface (130).

10. The pre-examination patient information gathering system as set forth in claim 1, further comprising:
a database (56) storing possible follow-up questions, the processor (50) selecting the follow-up questions from the possible follow-up questions stored in the database based on the received responses.

11. The pre-examination patient information gathering system as set forth in claim 1, further comprising:
a query input/output module (52) configured to validate the received responses.

12. The pre-examination patient information gathering system as set forth in claim 1, wherein the electronic processor (50) is further configured to prioritize patients for patient examination by a physician based on the received responses.

13. The pre-examination patient information gathering system as set forth in claim 1, wherein the electronic user interface further comprises:
a physiological sensor (70, 72, 74, 76, 78, 80) configured to autonomously sense a patient physiological parameter.

14. The pre-examination patient information gathering system as set forth in claim 13, wherein the electronic processor (50) is further configured to construct or select at least one follow-up question based on the patient physiological parameter sensed by the physiological sensor (70, 72, 74, 76, 78, 80).

15. The pre-examination patient information gathering system as set forth in claim 1, wherein the electronic processor (50) is further configured to define a clinical decision support system (CDSS) (90) configured to infer a potential medical condition based at least on the received responses.

16. A pre-examination patient information gathering method comprising:
presenting an initial set of questions (54) to a patient via an electronic user interface (30, 130) including a display (32) and at least one user input device (34, 36);
receiving responses to the initial set of questions from the patient via the electronic user interface;
electronically constructing or selecting follow-up questions based on the received responses;
presenting the follow-up questions to the patient via the electronic user interface; and
receiving responses to the follow-up questions from the patient via the electronic user interface.

17. The pre-examination patient information gathering method as set forth in claim 16, further comprising:
providing the patient with an incentive to utilize the electronic user interface (30, 130).

18. The pre-examination patient information gathering method as set forth in claim 17, wherein the providing comprises at least one of:
providing the patient with a pecuniary discount on the patient's medical insurance,
providing the patient with a coupon redeemable for a pecuniary benefit, and
providing the patient with reduced waiting time.

19. The pre-examination patient information gathering method as set forth in claim 16, wherein the electronic user interface (30) comprises a kiosk (22) disposed in a medical office (10), and the method further comprises:
unobtrusively sensing a patient physical parameter while the patient interacts with the kiosk (22).

20. A pre-examination patient information gathering method comprising:
arranging an electronic physiological sensor (70, 72, 74, 76) in a medical office (10) such that the electronic physiological sensor can sense a patient physiological parameter; and
autonomously operating the electronic physiological sensor to unobtrusively sense the patient physiological parameter.

21. The pre-examination patient information gathering method as set forth in claim 20, wherein the arranging comprises:
providing a user interface (30) for presenting the patient with questions and for receiving responses to the questions from the patient, the electronic physiological sensor (70, 72, 74, 76) autonomously operating to sense the patient physiological parameter during interaction of the patient with the user interface.

22. The pre-examination patient information gathering method as set forth in claim 21, wherein:
the user interface is selected from a group consisting of (i) an electronic user interface (30) disposed in the medical office and (ii) an operative combination of furniture, paper marked with the questions, and writing implement disposed in the medical office; and
the electronic physiological sensor includes at least one member of a group consisting of (i) a weight scale (70) arranged to weigh the patient, (ii) a camera (72, 74) arranged to view the patient, and (iii) a chemical sensor (76) arranged to detect a chemical emanating from the patient.

23. A pre-examination patient information gathering system comprising:
a user interface (30) for presenting the patient with questions and for receiving responses to the questions from the patient; and
an electronic physiological sensor (70, 72, 74, 76) arranged to unobtrusively sense a patient physiological parameter while the patient is interacting with the user interface.

24. The pre-examination patient information gathering system as set forth in claim 23, wherein the electronic physiological sensor includes at least one member of a group consisting of:
a weight scale (70) arranged to unobtrusively weigh the patient while the patient is interacting with the user interface (30);
a camera (72, 74) arranged to unobtrusively image the patient while the patient is interacting with the user interface; and
a chemical sensor (76) arranged to unobtrusively sense an airborne chemical or a chemical transmitted by contact while the patient is interacting with the user interface.
